(19) United States
(12) Patent Application Publication
(10) Pub. No.: US 2021/0205570 A1
(43) Pub. Date: Jul. 8, 2021

(54) MAGNETIC DAMPER AND ONE-WAY VALVE AND ANESTHESIA RESPIRATOR COMPRISING MAGNETIC DAMPER

(71) Applicant: GE PRECISION HEALTHCARE LLC, Wauwatosa, WI (US)

(72) Inventors: Yuyan ZHANG, Wuxi (CN); Qunfeng ZHANG, Wuxi (CN); Shuxian ZHAO, Wuxi (CN)

(21) Appl. No.: 17/136,256

(22) Filed: Dec. 29, 2020

(30) Foreign Application Priority Data
Jan. 3, 2020 (CN) 202010005799.9

(51) Int. Cl. A61M 16/20; A61M 16/01; A61M 16/08; A61M 39/24

(52) U.S. Cl. CPC A61M 16/201; A61M 16/01; A61M 2039/267; A61M 39/24; A61M 16/0891

(57) ABSTRACT

Embodiments of the present invention relate to a magnetic damper that can be used in a one-way valve of an anesthesia respirator, the magnetic damper including: a sealing element, capable of moving between a first position and a second position, wherein the one-way valve is opened when the sealing element is in the first position, and the one-way valve is closed when the sealing element is in the second position; a magnet, connected to the sealing element and used for driving the sealing element to move between the first position and the second position; and a coil, capable of inducing a current by means of movement of the magnet, wherein the current can produce a damping effect on the movement of the magnet. The embodiments of the present invention further relate to a one-way valve and an anesthesia respirator including the magnetic damper.

### CROSS-REFERENCE TO RELATED APPLICATIONS

This application claims the benefit of CN Patent Application No. 202010005799.9 filed on Jan. 3, 2020, the disclosure of which is herein incorporated by reference in its entirety.

### FIELD OF THE INVENTION

Embodiments of the present invention relate to the medical field, and particularly, to a magnetic damper that can be used in a one-way valve of an anesthesia respirator, as well as a one-way valve and an anesthesia respirator comprising the magnetic damper.

### BACKGROUND OF THE INVENTION

An anesthesia respirator is operating room equipment that is mainly used for providing support for gaseous anesthesia and respiratory management to a patient undergoing surgery. One of important functions of the anesthesia respirator is to maintain gases inhaled and exhaled by the patient at an appropriate flow and pressure.

A driving gas one-way valve (DCGV) is usually used in the anesthesia respirator to control the flow of a driving gas flowing to a bellow component. When the DCGV is opened, the driving gas can flow to the bellow component through the DCGV to push the bellow down and start to operate for respiration. During the process, it is important to keep the flow stable. An unstable flow would cause vibration or oscillation, and further cause pressure fluctuation in the patient airway or difficulty in exhaling.

Therefore, it is desired to improve the existing device to effectively solve or mitigate at least one of the currently existing problems.

### SUMMARY OF THE INVENTION

One aspect of embodiments of the present invention relates to a magnetic damper that can be used in a one-way valve of an anesthesia respirator, the magnetic damper comprising:

a sealing element, capable of moving between a first position and a second position, wherein the one-way valve is opened when the sealing element is in the first position, and the one-way valve is closed when the sealing element is in the second position;

a magnet, connected to the sealing element and used for driving the sealing element to move between the first position and the second position; and

a coil, capable of inducing a current by means of movement of the magnet, wherein the current can produce a damping effect on the movement of the magnet.

In the magnetic damper according to the embodiments of the present invention, optionally, the magnet is capable of reciprocating in a direction of gravity to drive the sealing element to move between the first position and the second position, and a weight of the magnet is designed so as to provide a threshold pressure required for opening the one-way valve.

In the magnetic damper according to the embodiments of the present invention, optionally, the magnet and the coil are arranged in a manner that enables the magnet to pass through the coil during movement.

In the magnetic damper according to the embodiments of the present invention, optionally, the coil is closed.

In the magnetic damper according to the embodiments of the present invention, optionally, the coil has two terminal leads, and the two terminal leads are short-circuited in a first mode of the one-way valve, and the two terminal leads are connected to a power source or another device in a second mode of the one-way valve.

In the magnetic damper according to the embodiments of the present invention, optionally, a current I can be induced in the coil when the magnet moves, the current I applies a force F to the magnet, a direction of the force F is opposite to a direction of movement of the magnet, and F=KBLIN, where K is a constant, B is a magnetic flux density of the magnet, L is a length of a single turn of wire of the coil, and N is the number of turns of the coil.

In the magnetic damper according to the embodiments of the present invention, optionally, the magnetic damper further comprises a former, wherein the coil is wound on the former.

In the magnetic damper according to the embodiments of the present invention, optionally, the magnetic damper further comprises a frame, wherein the former is mounted at the frame, and the frame is used for mounting to a portion of the one-way valve.

In the magnetic damper according to the embodiments of the present invention, optionally, the magnetic damper further comprises a housing, wherein the housing is provided with an inner space therein for accommodating other components in the magnetic damper, and the housing is further provided with a gas outlet in communication with the inner space, and when the one-way valve is opened, a gas can flow into the inner space from a chamber in the one-way valve and then flow out of the gas outlet.

Another aspect of the embodiments of the present invention relates to a one-way valve that can be used in an anesthesia respirator, the one-way valve comprising:

a valve body, provided with a vent thereon, wherein the one-way valve is opened when the vent is opened, and the one-way valve is closed when the vent is closed;

a sealing element, capable of moving between a first position and a second position, wherein the vent is opened when the sealing element is in the first position, and the vent is closed when the sealing element is in the second position;

a magnet, connected to the sealing element and used for driving the sealing element to move between the first position and the second position; and

a coil, capable of inducing a current by means of movement of the magnet, wherein the current can produce a damping effect on the movement of the magnet.

In the one-way valve according to the embodiments of the present invention, optionally, the sealing element comprises a silicone rubber sealing element attached below the magnet.

Another aspect of the embodiments of the present invention relates to an anesthesia respirator, comprising the magnetic damper or the one-way valve according to any one of the aforementioned embodiments.

### DETAILED DESCRIPTION OF THE DRAWINGS

The present invention will be described below in detail with reference to the embodiments illustrated in the drawings, in which:

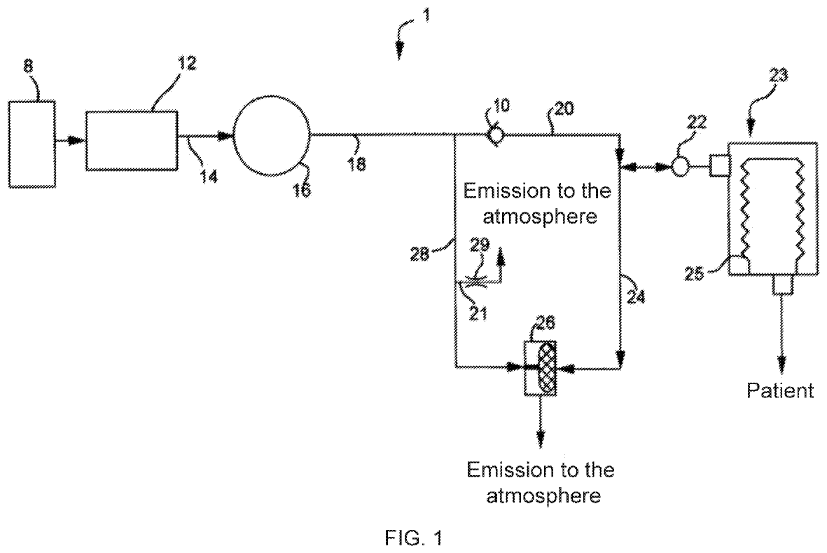

FIG. 1 shows a schematic view of a portion of an exemplary anesthesia respirator;

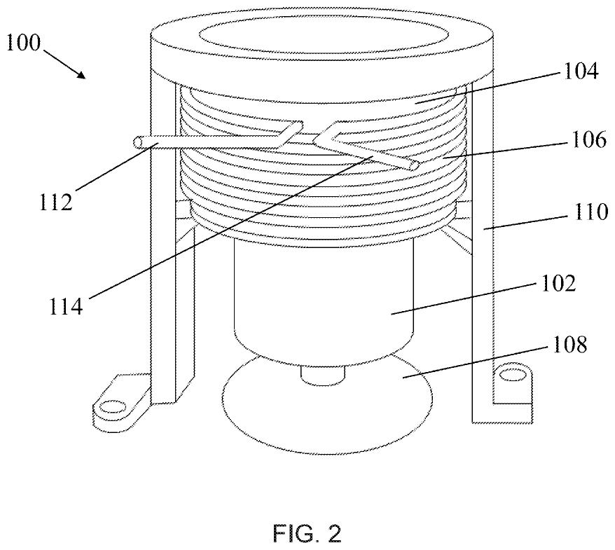

FIG. 2 shows a schematic perspective view of an exemplary magnetic damper that can be used in a one-way valve of an anesthesia respirator according to an embodiment of the present invention;

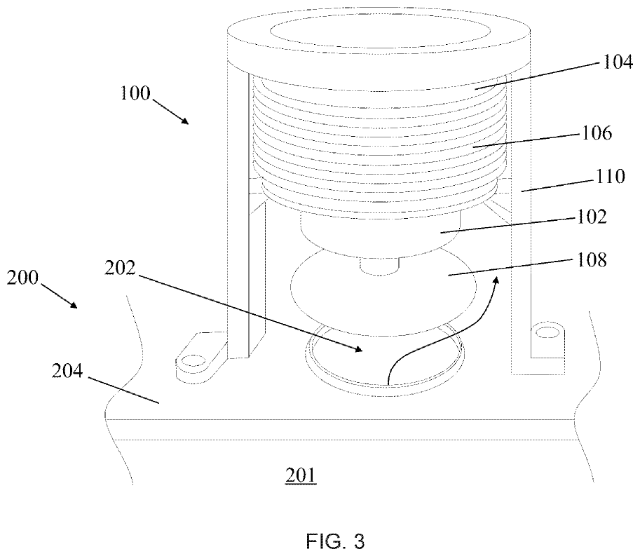

FIG. 3 shows a state in which the magnetic damper shown in FIG. 2 is mounted on an exemplary one-way valve and a sealing element therein is in a first position;

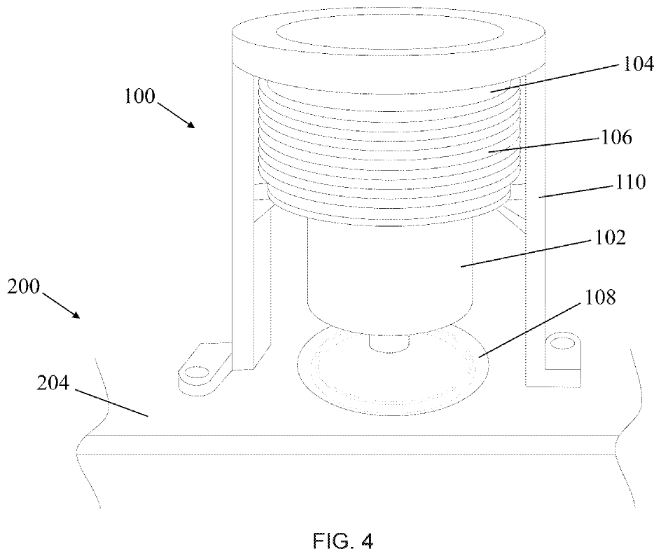

FIG. 4 shows a state in which the sealing element in the magnetic damper shown in FIG. 3 is in a second position; and

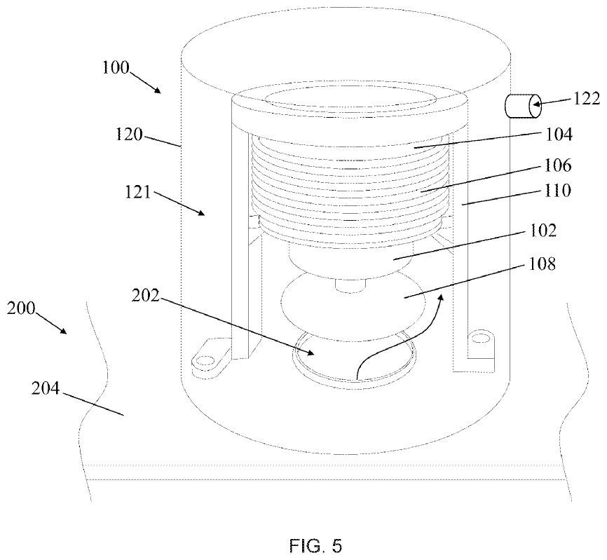

FIG. 5 shows a schematic perspective view of the magnetic damper including a housing, wherein the housing is represented in a transparent form to show the interior structure thereof.

### DETAILED DESCRIPTION OF THE PREFERRED EMBODIMENTS

Some embodiments of the present invention will be described below in more detail with reference to the drawings. Unless otherwise clearly defined herein, the meanings of scientific and technical terms used herein refer to meanings generally understood by those skilled in the art.

The terms “comprising”, “having”, and similar terms used herein mean that other items may also be included in the scope in addition to the items listed thereafter and their equivalents. The term “or” does not imply exclusion, but refers to the existence of at least one of the items mentioned, and encompasses the situation that a combination of the items mentioned exists. The term “and/or” includes any and all combinations of one or more of the items mentioned. “Some embodiments” mentioned herein means that a specific element (for example, feature, structure and/or characteristic) related to the present invention is included in at least one embodiment of the description and may or may not appear in other embodiments. In addition, it should be understood that the elements of the invention may be combined in any appropriate manner.

Herein, when a component is referred to as “provided on”, “mounted to” or “connected to” (or similar terms) another component, it can be directly provided on, mounted to or connected to the other component, or indirectly provided on, mounted to or connected to the other component by means of another intermediate element. In addition, the two components represented as “connected” or “interconnected” may be two independent components that are directly or indirectly connected together by means of a connecting device, or two components (namely, two portions of a whole) integrally formed together.

One aspect of embodiments of the present invention relates to a magnetic damper that can be used in a one-way valve of an anesthesia respirator. FIG. 1 shows a schematic view of a portion of an exemplary anesthesia respirator 1. As shown in FIG. 1, the anesthesia respirator 1 includes a gas source 8. The gas source 8 usually provides a pressurized gas at a pressure of about 50 psi, the gas source 8 is in communication with a source tube 14 by means of a main regulator 12, and the source tube 14 provides a respiratory gas of about 26 psi to a flow control valve 16. The flow control valve 16 is typically a proportional solenoid valve and controls the flow of the gas entering a tube 18. The tube 20 is in communication with the tube 18 and provides an inhalation branch to a respirator interface 22. A tube 24 provides an exhalation branch that serves to convey the gas from the respirator interface 22 to an exhaust valve 26. A one-way valve (check valve) 10 is located in the tube 20 to prevent the gas of the patient interface 22 from flowing into the tube 18 from the tubes 24 and 20 when the gas is exhaled.

The exhalation valve 26 controls the pressure and flow passing through the tube 24. The exhalation valve 26 is typically a diaphragm valve or balloon valve capable of controlling the pressure in the tube 24 according to a reference pressure. The reference control pressure is provided to the exhalation valve 26 by means of a pressure control tube 28. A flow restrictor 29 is provided on an exhaust tube 21 to provide control of emissions from the pressure control tube 28. When the pressure in the exhalation tube 24 exceeds the reference pressure in the tube 28, the gas is emitted from the exhalation tube 24 into the atmosphere. Thus, the pressure in the exhalation tube 24 is controlled by the reference pressure in the pressure control tube 28, and the reference pressure is controlled by the flow control valve 16.

The respirator interface 22 may be connected to a patient by means of a bellow component 23, or may be directly connected to the patient. In the application of connecting the respirator interface 22 to the patient by means of the bellow component 23, the tube 20 is in communication with an external chamber of a bellow 25 to actuate the bellow 25, and the respiratory tract of the patient is in communication with the interior of the bellow 25 and thus isolated from the gas in the respirator 8. Alternatively, in an ICU application, the bellow component 23 may be omitted, and the respirator interface 22 may be in direct communication with the respiratory tract of the patient, thereby directly providing the respiratory gas to the patient.

The tubes 18, 20, and 24 define a loop of the respirator in communication with the respirator interface 22. In most inhalation phases of the respiration of the patient, the gas flow is transported from the gas source 8 to the tubes 18 and 20 through the flow control valve 16 and finally arrives at the patient interface 22. In most exhalation phases of the respiration of the patient, the one-way valve 10 blocks the gas flow from the tube 20 to the tube 18, and the gas arrives at the exhalation valve 26 through the tube 24 and then is emitted to the atmosphere.

Typically, the one-way valve 10 is provided with a chamber therein for accommodating the gas, and provided with a vent thereon that can be closed or opened by a sealing element. When the vent is opened, the gas chamber in the one-way valve 10 is in communication with a rear gas path. When the vent is closed, the gas chamber in the one-way valve 10 is isolated from the rear gas path. In order to gently open and close the vent to prevent the pressurized gas flow flowing out of the vent from impacting the bellow 25 to cause vibration in the process of pushing the bellow 25 on the rear end, a damping structure may be provided on the sealing element of the vent. In some embodiments, the one-way valve 10 includes a hollow rectangular box structure, and the vent is provided on a top flat plate of the rectangular box structure.

FIGS. 2-5 show schematic perspective views of an exemplary magnetic damper that can be used in a one-way valve of an anesthesia respirator. As shown in FIGS. 2-5, the magnetic damper 100 includes a magnet 102, a coil 106 wound on a former 104, and a sealing element 108 connected to the magnet 102, and the magnetic damper is mounted on a top flat plate 204 adjacent to a vent 202 of a hollow rectangular box structure 200 (only a portion of the top flat plate 204 thereof provided with the vent 202 is shown in FIGS. 3-5) of the one-way valve. The sealing element 108 is attached to the magnet 102 such that the sealing element 108 is capable of moving between a first position and a second position along with the movement of the magnet 102. In the first position, as shown in FIG. 3, the sealing element 108 is away from the vent 202 to open the vent 202, thereby opening the one-way valve. When the vent 202 is opened, the gas flow can flow out of a chamber 201 in the one-way valve by means of the vent 202. In the second position, as shown in FIG. 4, the sealing element 108 covers the vent 202 provided on the one-way valve to close the vent 202, thereby closing the one-way valve. In this manner, the opening and closing of the one-way valve can be controlled by means of controlling the movement of the magnet 102 to implement the function of the one-way valve.

In some embodiments, the coil 106 is a copper coil. In some embodiments, the sealing element 108 is a silicone rubber seal ring. In some embodiments, the former 104 is assembled at a frame 110, and the frame 110 can be mounted on the top flat plate 204 on two sides of the vent 202 of the one-way valve to ensure that the sealing element 108 can completely cover the vent 202 in the second position. In some embodiments, the magnetic damper 100 further includes a housing 120. The housing 120 is provided with an inner space 121 therein for accommodating other components in the magnetic damper 100, and the housing 120 is further provided with a gas outlet 122 in communication with the inner space 121 to allow the gas to flow toward the rear gas path from the inner space 121. The housing 120 can be hermetically mounted on the top flat plate 204 of the one-way valve, accommodate other components in the magnetic damper 100 therein, and cause the vent 202, when opened, to be in communication with the inner space 121 of the housing 120. In this way, when the vent 202 is opened, the gas can flow into the inner space 121 of the housing 120 from the chamber 201 in the one-way valve by means of the vent 202 and then flow out of the gas outlet 122.

The magnet 102 is capable of reciprocating in a direction of gravity to drive the sealing element 108 to move between the first position and the second position. The weight of the magnet 102 is designed so as to provide a threshold pressure required for opening the one-way valve. In other words, the gravity G of the magnet 102 can be applied to the sealing element 108 so as to seal the vent 202 of the one-way valve by gravity. In some embodiments, the magnet 102 seals the vent 202 of the one-way valve by gravity, such that the gas pressure in a front gas path of the one-way valve is equal to the sum of the gas pressure in the rear gas path of the one-way valve and the gravity of the magnet 102. For example, as shown in FIG. 1, the gas pressure F₂₈ in the front gas path (the tube 28) of the one-way valve 10 is equal to the sum of the gas pressure F₂₀ in the rear gas path (the tube 20) of the one-way valve 10 and the gravity G of the magnet 102, that is, F₂₈=F₂₀+G. In this way, a pressure difference G exists between two sides of the exhalation valve 26. Because the pressure difference G exists, a diaphragm in the exhalation valve 26 would be in a closed state. The aforementioned configuration enables the driver gas to automatically close the exhalation valve 26 during pressurization and driving, thereby avoiding leakage of the driving gas. In addition, the magnet 102 and the coil 106 are arranged in a manner that enables the magnet 102 to pass through the coil 106 during movement.

In some embodiments, the coil 106 is made closed (as shown in FIGS. 3-5). In some embodiments, the coil 106 is made open looped (as shown in FIG. 2), which has two terminal leads 112 and 114. In at least one mode (for example, a first mode) of the one-way valve, the two terminal leads 112 and 114 thereof are short-circuited, without being connected to a power source and powered. In at least one mode (for example, a second mode) of the one-way valve, the two terminal leads 112 and 114 thereof are connected to a power source or another device, so as to provide further performance control according to specific needs. For example, in a maintenance mode of the one-way valve, the two terminal leads 112 and 114 of the coil 106 can be connected to an external power source, so as to control the one-way valve to be continuously open or closed for maintenance in a period of time according to needs.

The working principle of the magnetic damper 100 may be substantially the same as that of a voice coil motor. When the magnet 102 moves upward or downward, the coil 106 would cut movement of a magnetic field generated by the magnet 102. A voltage is induced at two ends of a conductor in the coil 106. The magnitude E of the voltage can be represented by the following equation:

E=KBLvN,

where K is a constant, B is a magnetic flux density of the magnet 102, L is a length of a single turn of wire of the coil 106, v is a velocity of movement of the magnet 102 relative to the coil 106, and N is the number of turns of the coil 106.

According to principle of Lorentz force, a force F is applied to the magnet 102 when a current carrying conductor (the coil 106) is placed in the magnetic field of the magnet 102. The direction of the force F is opposite to the direction of movement of the magnet 102. The force F can be considered as a damping force generated by the magnetic damper 100. The magnitude of the force F can be represented by the following equation:

F=KBLIN,

where I is a current in the coil 106, and similar to the above equation, K is a constant, B is a magnetic flux density of the magnet 102, L is a length of a single turn of wire of the coil 106, and N is the number of turns of the coil 106.

The damping coefficient c of the magnetic damper 100 can be represented by the following equation:

c=−F/v=(−KBLIN×KBLN)/(I×R)=−(KBLN)²/R.

It should be noted that the above equation is only an estimation of the force F in some cases and does not constitute a limitation of the magnetic damper and the associated device structure thereof in any of the aforementioned embodiments of the present invention.

The magnetic damper and the one-way valve or the anesthesia respirator including the magnetic damper in the embodiments of the present invention can ensure continuous and smooth flow distribution and prevent vibration. The parts used in the magnetic damper are low-cost and readily available, and do not have high precision requirement in the manufacturing process, thereby providing an alternative solution for expensive single source air buffers. In addition, moving components in the magnetic damper do not need sealing and lubrication, and can provide a damping function with zero friction, without mechanical wear due to magnetic forces. The magnetic damper can also provide a precisely controlled damping force. The factors determining the damping force are mainly the coil resistance and magnetic flux density, which are easily controlled during manufacturing.

The purpose of providing the above specific embodiments is to facilitate understanding of the content disclosed in the present invention more thoroughly and comprehensively, but the present invention is not limited to these specific embodiments. Those skilled in the art should understand that various modifications, equivalent replacements, and changes can also be made to the present invention and should be included in the scope of protection of the present invention as long as these changes do not depart from the spirit of the present invention.

1. A magnetic damper that can be used in a one-way valve of an anesthesia respirator, the magnetic damper comprising:
a sealing element, capable of moving between a first position and a second position, wherein the one-way valve is opened when the sealing element is in the first position, and the one-way valve is closed when the sealing element is in the second position;
a magnet, connected to the sealing element and used for driving the sealing element to move between the first position and the second position; and
a coil, capable of inducing a current by means of movement of the magnet, wherein the current can produce a damping effect on the movement of the magnet.

2. The magnetic damper according to claim 1, wherein the magnet is capable of reciprocating in a direction of gravity to drive the sealing element to move between the first position and the second position, and a weight of the magnet is designed so as to provide a threshold pressure required for opening the one-way valve.

3. The magnetic damper according to claim 1, wherein the magnet and the coil are arranged in a manner that enables the magnet to pass through the coil during movement.

4. The magnetic damper according to claim 1, wherein the coil is closed.

5. The magnetic damper according to claim 1, wherein the coil has two terminal leads, and the two terminal leads are short-circuited in a first mode of the one-way valve, and the two terminal leads are connected to a power source or another device in a second mode of the one-way valve.

6. The magnetic damper according to claim 1, wherein a current I can be induced in the coil when the magnet moves, the current I applies a force F to the magnet, a direction of the force F is opposite to a direction of movement of the magnet, and F=KBLIN, where K is a constant, B is the magnetic flux density of the magnet, L is the length of a single turn of wire of the coil, and N is the number of turns of the coil.

7. The magnetic damper according to claim 1, further comprising a former, wherein the coil is wound on the former.

8. The magnetic damper according to claim 7, further comprising a frame, wherein the former is mounted at the frame, and the frame is used for mounting to a portion of the one-way valve.

9. The magnetic damper according to claim 1, further comprising a housing, wherein the housing is provided with an inner space therein for accommodating other components in the magnetic damper, and the housing is further provided with a gas outlet in communication with the inner space, and when the one-way valve is opened, a gas can flow into the inner space from a chamber in the one-way valve and then flow out of the gas outlet.

10. A one-way valve that can be used in an anesthesia respirator, the one-way valve comprising:
a valve body, provided with a vent thereon, wherein the one-way valve is opened when the vent is opened, and the one-way valve is closed when the vent is closed;
a sealing element, capable of moving between a first position and a second position, wherein the vent is opened when the sealing element is in the first position, and the vent is closed when the sealing element is in the second position;
a magnet, connected to the sealing element and used for driving the sealing element to move between the first position and the second position; and
a coil, capable of inducing a current by means of movement of the magnet, wherein the current can produce a damping effect on the movement of the magnet.

11. The one-way valve according to claim 10, wherein the sealing element comprises a silicone rubber sealing element attached below the magnet.

12. An anesthesia respirator, comprising the magnetic damper according to claim 1.

13. An anesthesia respirator, comprising the magnetic damper according to claim 10.
